(19) United States
(12) Patent Application Publication
(10) Pub. No.: US 2004/0153131 A1
(43) Pub. Date: Aug. 5, 2004

(54) Apparatus and method for hair retention and regeneration

(76) Inventor: John A. Yorke, Utica, NY (US)

Correspondence Address: ARLEN L. OLSEN, SCHMEISER, OLSEN & WATTS, 3 LEAR JET LANE, SUITE 201, LATHAM, NY, 12110, US

(21) Appl. No.: 10/358,441

(22) Filed: Feb. 4, 2003

(51) Int. Cl. A61N 5/02

(52) U.S. Cl. 607/91; 607/89; 607/90

(57) ABSTRACT

An apparatus and method for regenerating and retaining hair wherein one or more regions of the scalp of a human head may be selectively and repeatedly exposed to low-level light (“LLL”) having a wavelength between about 400 nm and about 1000 nm and delivering an irradiance between about 100 mJ/cm² and about 900 mJ/cm² over variable exposure times. The LLL is emitted from a light source disposed upon a head covering for covering the scalp of a human head, and is directed toward the scalp when the head covering is worn upon the head. The head covering may be worn alone in one of several embodiments, or it may be disposed within one of several secondary means for covering a human head. The light source is chosen from the group consisting of light-emitting diodes, light-emitting diode arrays, laser diodes, laser diode arrays, and any combination thereof. The light source is operationally connected to means for selecting regions of the scalp to be exposed to LLL, means for selecting LLL wavelengths, means for selecting LLL exposure durations, means for selecting LLL irradiances, and means for selecting the emission of either continuous or pulsed LLL. A photoelectric cell is operationally connected to the head covering to deactivate the light source whenever the head is withdrawn from the head covering.

### BACKGROUND OF THE INVENTION

1. Technical Field

The present invention relates to a method and apparatus for retaining and regenerating hair.

2. Related Art

Alopecia (hair loss) is a major concern for the adult population. Expenditures for hair restoration products and treatments for hair loss represent a major component of the multibillion-dollar cosmetic industry in the United States. Examples of techniques for hair retention and regeneration include the use of topical drug therapies such as Minoxidil (Rogaine®) or oral drug therapies such as Finasteride (Propecia®), hair transplantation, hair weaving, the use of hairpieces, the application of hair thickening sprays and shampoos, and the fashioning of coiffures which distribute hair to cover balding regions of the scalp.

There is a need for a hair retention and regeneration technique that neither involves pharmacologic or surgical intervention nor cosmetic simulation, thereby avoiding the side effects of pharmacologic intervention, the discomfort of surgical intervention, and the synthetic appearance of a cosmetic appliance.

### SUMMARY OF THE INVENTION

The present invention is an apparatus for regenerating and retaining hair, comprising:

a head covering for covering a human head; and

a light source operatively connected to said head covering, wherein said light source is directed toward the scalp when the head covering is worn, to selectively and repeatedly expose one or more regions of the scalp in need of hair retention or regeneration to low-level light (“LLL”) having a wavelength between about 400 nm and about 1000 nm and delivering an irradiance between about 100 mJ/cm² and about 900 mJ/cm², over variable durations of exposure, in either a continuous or pulsed mode.

Said head covering may be worn alone in one of several embodiments, or it may disposed within one of several secondary head coverings for covering a human head. Said light source is chosen from the group consisting of light emitting diodes, light emitting diode arrays, laser diodes, laser diode arrays, and any combination thereof. Said light source is operably connected to a control module containing means for selecting regions of the scalp to be exposed to LLL, means for selecting LLL wavelengths, means for selecting LLL exposure durations, means for selecting LLL irradiances, and means for selecting the emission of either continuous or pulsed LLL.

A photoelectric cell is operably attached to said head covering to deactivate said light source whenever the head is withdrawn from said head covering. Depending on its embodiment, a pattern emission selector of said control module, which selects regions of the scalp to be exposed to LLL, may include icons, appearing upon either tiles displayed on a computer display device that is a component of said control module, or keys of a keyboard or keypad that are components of said control module, or buttons disposed on a surface of said control module, which icons depict degrees of hair loss comprising, inter alia, either the Hamilton scale or the Ludwig scale, or both.

The present invention is also a method for regenerating and retaining hair, comprising the application of LLL to at least one region of the scalp of a human head in need of hair retention or regeneration, said application of LLL being emitted from a light source chosen from the group consisting of light emitting diodes, light emitting diode arrays, laser diodes, laser diode arrays, and any combination thereof, disposed upon a head covering for covering a human head, and said light source operationally connected to means for selecting regions of the scalp in need of hair retention or regeneration to be exposed to LLL, means for selecting LLL wavelengths, means for selecting LLL exposure durations, means for selecting LLL irradiances, and means for selecting the emission of either continuous or pulsed LLL.

The present invention provides a new and useful apparatus and method for retaining and regenerating hair without the inconvenience, unpleasant side effects, pain, and poor appearance associated with prevailing hair retention and regeneration techniques. Embodiments of the apparatus for hair retention and regeneration are adaptable to salon settings, as well as portable and home use settings, where the method for hair retention and regeneration may be self-administered.

### BRIEF DESCRIPTION OF THE DRAWINGS

For a better understanding of the invention, reference may be had to the accompanying drawings in which:

FIG. 1 is a schematic representation of a first embodiment of a head covering of the invention.

FIG. 2 is a schematic representation of a first embodiment of a head covering of the invention worn upon a human head.

FIG. 3 is a schematic representation of a second embodiment of a head covering of the invention.

FIG. 4 is a schematic representation of a light-emitting diode, a light-emitting diode array, a laser diode, and a laser diode array.

FIG. 5 is a schematic representation of a first embodiment of a headpiece of the invention.

FIG. 6 is a schematic representation of a second embodiment of a headpiece of the invention.

FIG. 7 is a schematic representation of a first embodiment of the hair retention and regeneration apparatus comprising the invention.

FIG. 8 is a schematic representation of a second embodiment of the hair retention and apparatus comprising the invention.

FIG. 9 is a representation of the pictographs of the Hamilton scale for assessing male alopecia, shown as icons on schematic tiles, keys or buttons.

FIG. 10 is a representation of the pictographs of the Ludwig scale for assessing female alopecia, shown as icons on schematic tiles, keys or buttons.

FIG. 11 is a perspective and sectional view of a disposition of light-emitting diodes, light emitting diode arrays, laser diodes, and laser diode arrays upon an embodiment of the headpiece of the invention that is disposed within an embodiment of a secondary head covering, thereby forming a hood.

FIG. 12 is a perspective view of an embodiment of the invention schematically illustrating a disposition of conductors connecting an embodiment of the control module of the invention to an embodiment of the headpiece of the invention.

FIG. 13 is a perspective view of a stationary embodiment of the invention.

FIG. 14 is a perspective view of a portable embodiment of the invention.

FIG. 15 is plan view of a portable embodiment of the invention in its folded or collapsed position for carriage or storage.

FIG. 16 is a perspective view of a portable embodiment of the invention comprising an embodiment of the headpiece of the invention disposed within a secondary head covering in the form of a helmet, and schematically illustrating a hand-held embodiment of the control module of the invention.

### DETAILED DESCRIPTION OF THE INVENTION

As used herein, the term “hair regeneration” includes the term hair restoration.

As used herein, the term “hair retention” includes the terms hair conservation and hair preservation.

As used herein, the term “wireframe mesh” means a mesh woven of, or formed by interlacing, strands of such materials as metal, plastic, or synthetic fibers, such as nylon, rayon, or polyester, or natural fibers, such as cotton., or any combination thereof.

As used herein, the term “low-level light” (“LLL”) means ordinary or coherent light having a wavelength in the range of about 400 nm to about 1000 nm and having an irradiance at its point of contact with a bodily tissue, such as the skin or the scalp, between about 100 mJ/cm² and about 900 mJ/cm².

As used herein, the term “low-level light therapy” (“LLLT”) means, inter alia, the photobiostimulation of a bodily tissue, such as the skin or scalp, by exposure of said bodily tissue to low-level light (“LLL”).

As used herein the term “continuous” low-level light (“LLL”) means continuously emitted or radiated LLL, or temporally uninterrupted emission or radiation of LLL.

As used herein the term “pulsed” low-level light (“LLL”) means, inter alia, pulsed, flickering, interrupted, or otherwise discontinuous emission or radiation of LLL.

As used herein the term “emission mode” refers to the emission of LLL as either continuous LLL or pulsed LLL.

As used herein, the word “computer” means, inter alia, a microprocessor, server, desktop computer, laptop computer, personal digital assistant or other form of hand-held or portable computer.

As used herein, the term “data entry device” means, inter alia, a keyboard, keypad, stylus-based data entry device or a touch-based data entry device, such as a touchpad.

As used herein, the term “computer display” device means, inter alia, a computer monitor, liquid crystal display panel, television set or printer.

As used herein, the term “emission pattern” means the two-dimensional surface shape of a bodily tissue, such as the skin or the scalp, and the area contained therein, which is exposed to LLL by the invention.

As used herein, the term “emission pattern selector” refers to a device for confining LLL to a selected emission pattern.

As used herein, the term “photoelectric cell” means a photosensitive device such as a photodiode, phototransistor, photoconductive or photovoltaic cell or any combination or plurality thereof.

As used herein the term “conductors” means electrically insulated or non-insulated metal wires, fiberoptic cables or any other means for transmitting electric power.

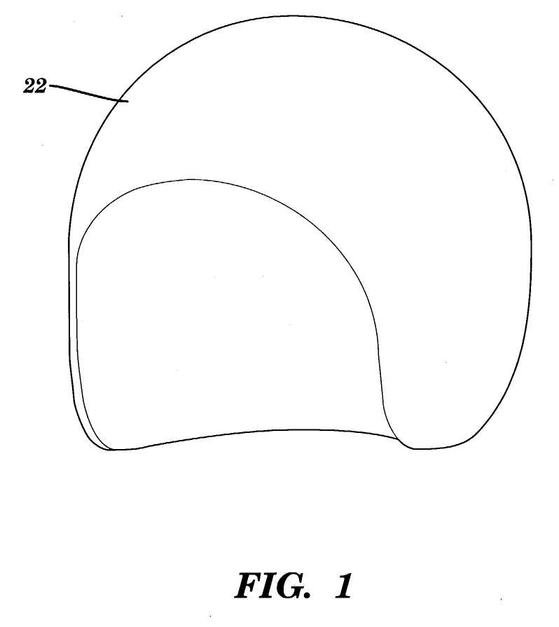

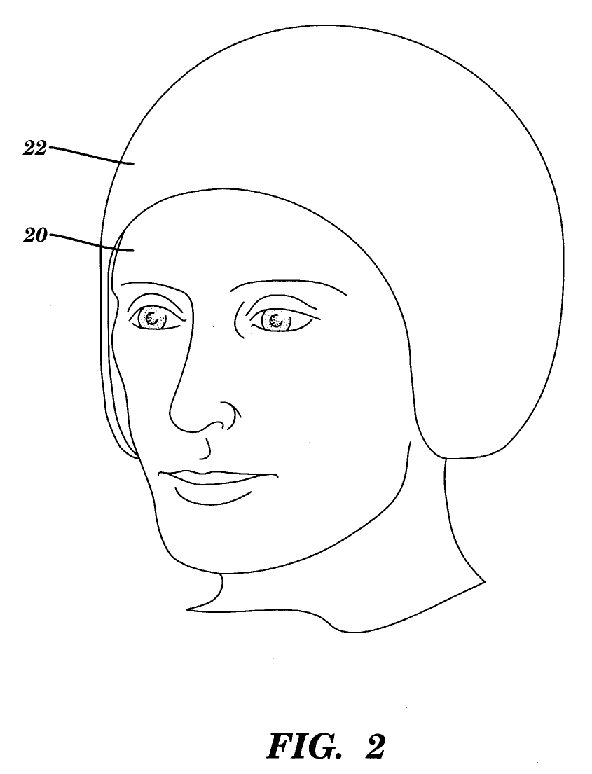

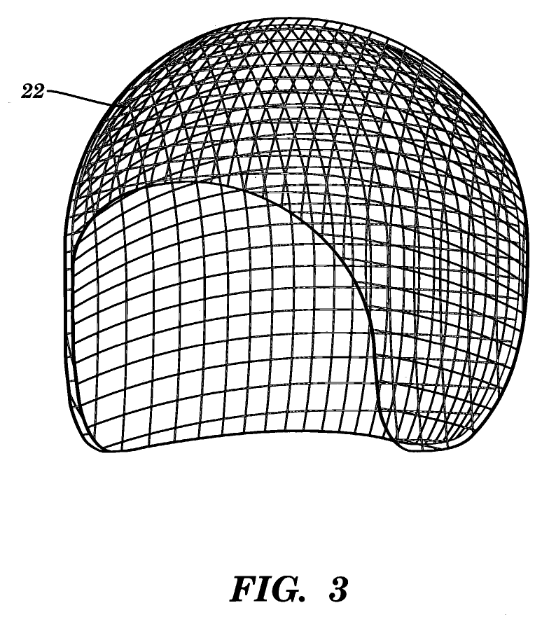

Referring now to the drawings in which like parts are designated by like numerals in the various views, FIG. 1 illustrates a first embodiment of a head covering 22 of the invention wherein the head covering is fashioned out of plastic. FIG. 2 illustrates the first embodiment of the head covering 22 of the invention shown in FIG. 1 worn upon a human head 20. FIG. 3 illustrates a second embodiment of a head covering 22 of the invention, wherein the head covering is fashioned out of a wireframe mesh. In either the constitutive material of the embodiment of the head covering shown in FIG. 1 or the constitutive material of the embodiment of the head covering shown in FIG. 3, the head covering 22 may be fashioned into any geometric form, including, but not limited to, a spatial configuration generally conforming to the shape of a human head, or any rigid, semi-rigid or flexible device for covering a human head, such as a helmet, hat, cap, hood, dome, cowl, or any sheet, such as a handkerchief.

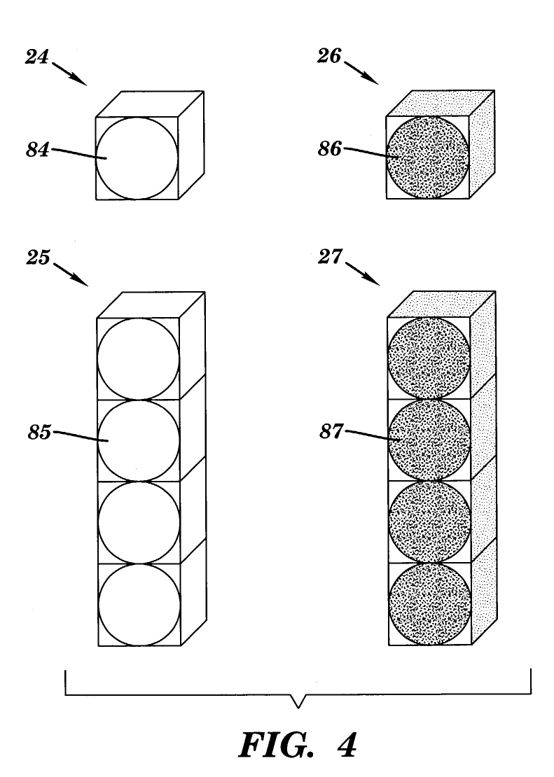

FIG. 4. is a schematic representation of a light-emitting diode (“LED”) 24, a LED array 25, a laser diode 26, and a laser diode array 27, each of which is shown as a rectangular parallelepiped, having an active, light-emitting face, schematically representing a single LED face 84, a single laser diode face 86, a representative LED array face 85, and a representative laser diode array face 87.

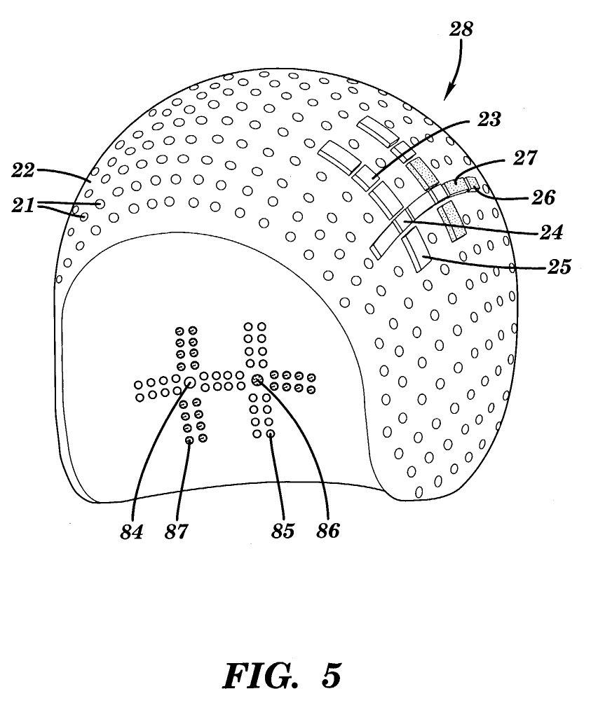

FIG. 5 illustrates a first embodiment of a headpiece 28 of the invention, comprising a head covering 22, as shown in FIG. 1, with multiple-perforations 21, upon whose external surface are disposed a photoelectric cell 23 and a representative combination of an inwardly directed LED 24, LED array 25, laser diode 26, and laser diode array 27, for the administration of LLL, having controllable wavelengths, irradiances, exposure durations, emission patterns, and emission modes, more fully described infra.

In FIG. 5, the inwardly directed LED 24, LED array 25, laser diode 26, and laser diode array 27, are shown as discrete rectangular parallelepipeds, corresponding to the LED 24, LED array 25, laser diode 26, or laser diode array 27, shown in FIG. 4. Each LED 24, LED array 25, laser diode 26, and laser diode array 27 shown in FIG. 5 has a respectively corresponding single LED face 84, single laser diode face 86, LED array face 85, and laser diode array face 87, all facing inwardly for radiant emission through one or more of the perforations 21 of the headpiece 28.

As shown in FIG. 5, when the representative combination of an LED 24, LED array 25, laser diode 26, and laser diode array 27 is viewed from within the headpiece 28, all that is visible are light sources appearing through the perforations 21 corresponding respectively to light emissions from the single LED face 84, single laser diode face 86, LED array face 85, and laser diode array face 87, of each externally disposed LED 24, LED array 25, laser diode 26, and laser diode array 27.

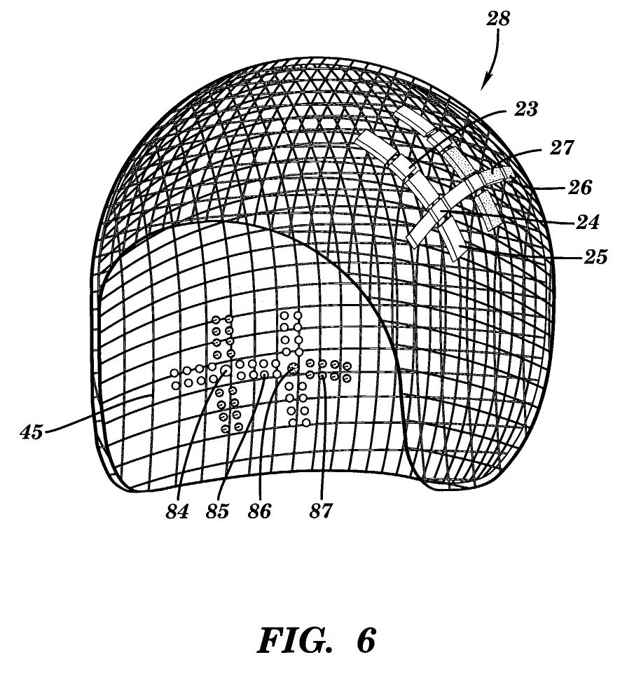

FIG. 6 illustrates a second embodiment of a headpiece 28 of the invention, comprising a head covering 22 as shown in FIG. 3, upon whose external surface are disposed a photoelectric cell 23 and a representative combination of an inwardly directed LED 24, LED array 25, laser diode 26, and laser diode array 27, for the administration of LLL, having controllable wavelengths, irradiances, exposure durations, emission patterns, and emission modes, more fully described infra.

In FIG. 6, the inwardly directed LED 24, LED array 25, laser diode 26, and laser diode array 27, are shown as discrete rectangular parallelepipeds, corresponding to the LED 24, LED array 25, laser diode 26, and laser diode array 27, shown in FIG. 4. Each LED 24, LED array 25, laser diode 26, or laser diode array 27 shown in FIG. 6 has a respectively corresponding single LED face 84, single laser diode face 86, LED array face 85, and laser diode array face 87, all facing inwardly for radiant emission through the interstices 45 of the headpiece 28.

As shown in FIG. 6, when the LED 24, LED array 25, laser diode 26, and laser diode array 27, are viewed from within the headpiece 28, all that is visible are their respectively corresponding light-emitting faces, 84, 85, 86, 87, either framed by the interstices 45 or crossed by the strands of which the second embodiment of the headpiece 28 is constructed.

Tissue exposed to LLL emitted by the embodiments of the headpiece 28 of the invention, as shown in FIG. 5 and FIG. 6, is subjected to photobiostimulation (“PBS”) which activates energy processes in intracellular mitochondria, wavelength-selective intracellular organelles, and other intracellular constituents such as chromophores and cytochrome systems.

In the case of retention and regeneration of the hair of the scalp, PBS is believed to occur between the endothelial cell of the inner root hair follicle sheath wall and the epithelial cuticle cells of the hair shaft. The net effects of the PBS generated by the LLL emitted from the headpiece 28 of the invention is to maintain hair shafts in the anagen stage of their growth cycle, reverse the deterioration of hair shafts, accelerate healthy morphological transformation in the catagen stage of the growth cycle of hair shafts, and to shorten or negate the telogen stage of the growth cycle of hair shafts.

Accordingly, LLLT of the scalp is a novel means of reducing hair loss and stimulating hair growth in individuals with alopecia. The practical advantages of using LLL for the treatment of alopecia include the avoidance of the undesirable side effects associated with the use of topical or oral drugs, the avoidance of the inconvenience and poor cosmesis offered by hair-thickening sprays and shampoos, the avoidance of the transience of a cosmetic coiffure, the avoidance of the pain and distasteful appearance associated with hair transplantation, and the avoidance of the synthetic appearance of a hairpiece and the embarrassment associated with its detection.

Combinations of LEDs, LED arrays, laser diodes, and laser diode arrays offer useful sources of LLL for PBS of the scalp. Such combinations of LEDs, LED arrays, laser diodes, and laser diode arrays can be configured to generate PBS within the scalp, without significant heating of the scalp and without risk to the unprotected eye. Additionally, such combinations of LEDs, LED arrays, laser diodes, and laser diode arrays can be arranged and configured to emit LLL targeting different locations of the scalp and to emit LLL confined to patterns of variable area in different locations of the scalp

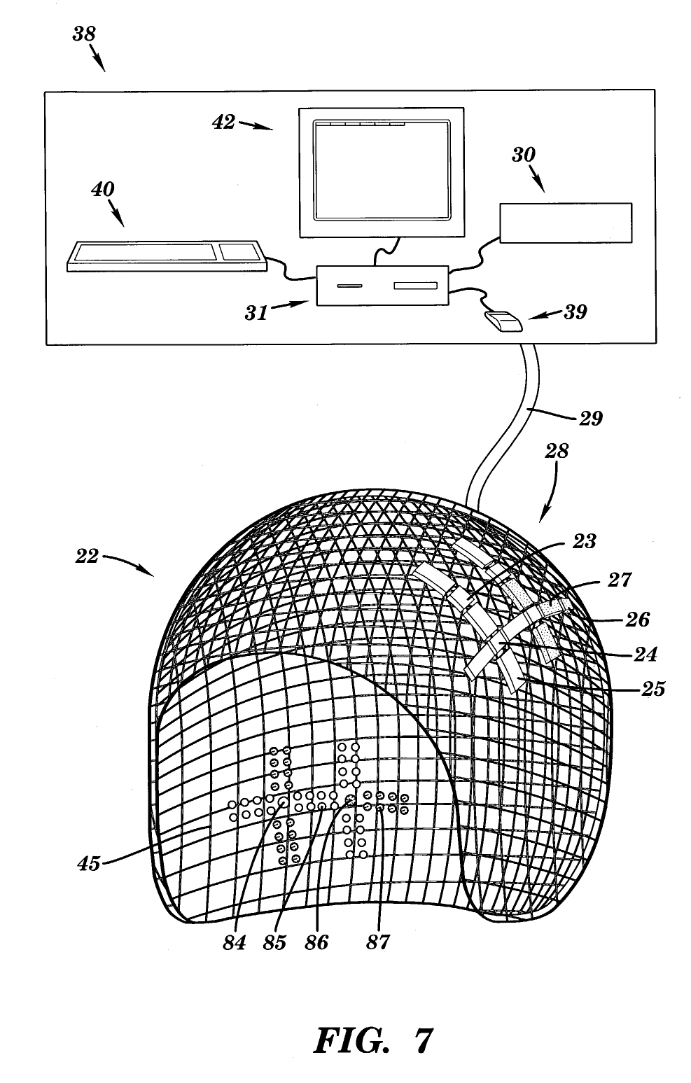

FIG. 7. illustrates a first embodiment of the invention, formed by using conductors 29 to operably connect the first embodiment of the headpiece 28 of the invention to a control module, 38, comprising at least one computer 31 operably connected to a power source 30 and to computer input and output devices, such as a pointing device 39, a data entry device 40, and a computer display device 42, for controlling the wavelength, irradiance, duration of exposure, emission pattern, and emission mode, used in the administration of LLL to one or more regions of the scalp.

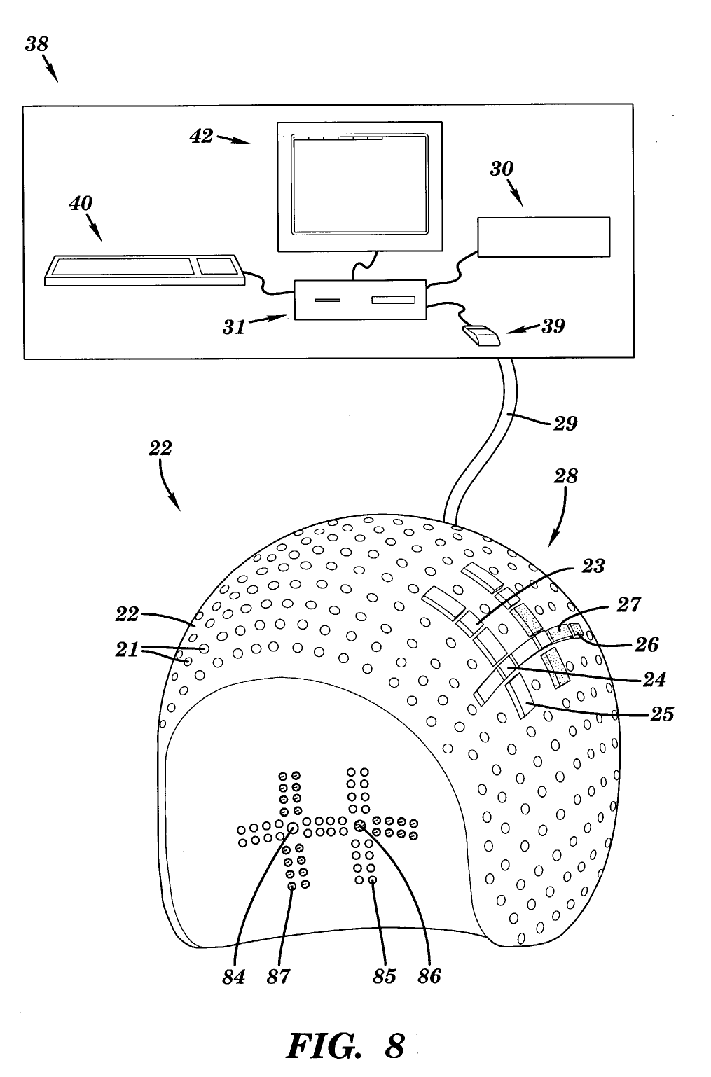

FIG. 8. illustrates a second embodiment of the invention, formed by using conductors 29 to operably connect the second embodiment of the headpiece 28 of the invention to a control module 38 comprising at least one computer 31 operably connected to a power source 30 and to input and devices, such as a pointing device 39, a data entry device 40, and a computer display device 42, for controlling the wavelength, irradiance, duration of exposure, emission pattern, and emission mode, used in the administration of LLL to one or more regions of the scalp.

The power source 30 may be a direct current power source such as a battery, or a computer-controllable device whose input is an alternating current power source, such a as a conventional electric power outlet. The photoelectric cell 23 is also operably connected to the power source 30 as a means for turning off the power source 30 whenever the head is withdrawn from the headpiece 28 of the invention.

The control module 38 further includes an emission pattern selector, providing means for confining the LLL emitted by the headpiece 28 of the invention to patterns of hair loss, conforming to the patterns of hair loss actually present upon the scalp of any subject. In addition to providing means for confining the LLL emitted by the headpiece 28 to patterns of hair loss present on the scalp on a subject-by-subject basis, the emission pattern selector provides means for confining the LLL emitted by the headpiece 28 to commonly observed patterns of hair loss well known in the hair replacement arts

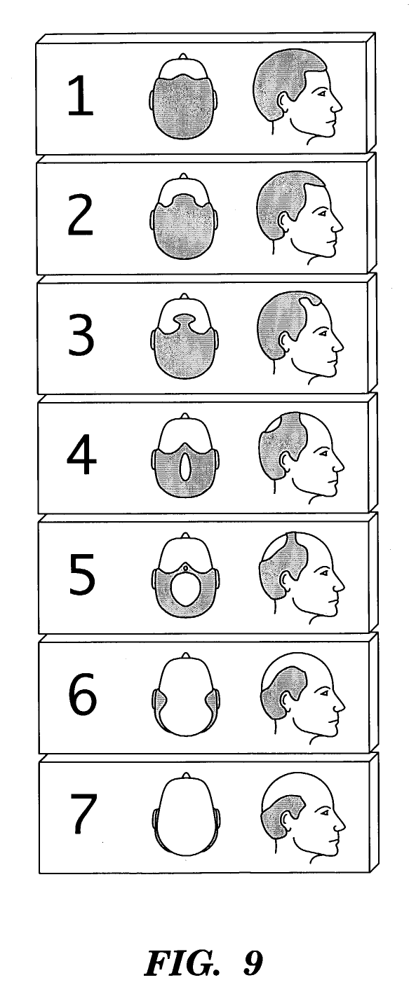

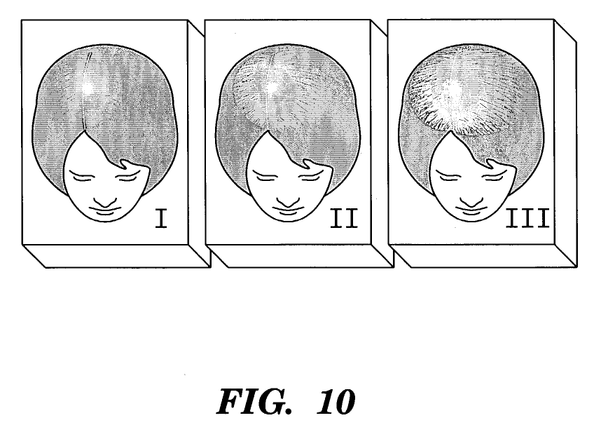

In a first embodiment of the emission pattern selector, in addition to providing means of confining the LLL emitted by the headpiece 28 to patterns of hair loss present on the scalp on a subject-by-subject basis, the screen of the computer viewing device 40 displays an operator's interface comprising arrays of symbolic tiles. As shown in FIG. 9, in a first array of these tiles, an icon appearing on the face of each tile depicts a pattern of hair loss replicating one of the states of hair loss of the Hamilton scale, a standardized pictographic scale of degrees and patterns of hair loss among men, well known in the hair retention, regeneration and replacement arts. As shown in FIG. 10, in a second array of tiles, an icon appearing on the face of each tile depicts a pattern of hair loss replicating one of the states of hair loss of the Ludwig scale, a standardized pictographic scale of degrees and patterns of hair loss among women, also well known in the hair retention, regeneration and replacement arts. By pointing and clicking upon a tile with a cursor controlled by a computer pointing device 39, the operator selects means for activating a subcombination of any combination of LEDs 24, LED arrays 25, laser diodes 26, and laser diode arrays 27, disposed upon the headpiece 28 of the invention, which subcombination confines the emission of the LEDs 24, LED arrays 25, laser diodes 26, and laser diode arrays 27 to the pattern of hair loss represented by the icon on the selected tile.

In a second embodiment of the emission pattern selector, in addition to providing means of confining the LLL emitted by the headpiece 28 to patterns of hair loss present on the scalp on a subject-by-subject basis, the data entry device 40, such as a keyboard or keypad includes sets of symbolic keys. As shown in FIG. 9, in a first set of keys, an icon appearing on the face of each key depicts a pattern of hair loss replicating one of the states of hair loss of the Hamilton scale, a standardized pictographic scale of degrees and patterns of hair loss among men, well known in the hair retention, regeneration and replacement arts. As shown in FIG. 10, in a second set of keys, an icon appearing on the face of each key depicts a pattern of hair loss replicating one of the states of hair loss of the Ludwig scale, a standardized pictographic scale of degrees and patterns of hair loss among women, also well known in the hair retention, regeneration and replacement arts. By striking an icon-bearing key, the operator selects means for activating a subcombination of any combination of the LEDs 24 , LED arrays 25, laser diodes 26, and laser diode arrays 27, disposed upon the headpiece 28 of the invention, which subcombination confines the emission of the LEDs 24, LED arrays 25, laser diodes 26, and laser diode arrays 27 to the pattern of hair loss represented by the icon on the selected key.

In a third embodiment of the of the pattern emission selector, in addition to providing means of confining the LLL emitted by the headpiece 28 to patterns of hair loss present on the scalp on a subject-by-subject basis, the control module 38 includes a surface having arrays of buttons. As shown in FIG. 9, in a first array of buttons, an icon appearing on the face of each button depicts a pattern of hair loss replicating one of the states of hair loss of the Hamilton scale, scale, a standardized pictographic scale of degrees and patterns of hair loss among men, well known in the hair retention, regeneration and replacement arts. As shown in FIG. 10, in a second array of buttons, an icon appearing on the face of each button depicts a pattern of hair loss replicating one of the states of hair loss of the Ludwig scale, a standardized pictographic scale of degrees and patterns of hair loss among women, also well known in the hair retention, regeneration and replacement arts. By pressing an icon-bearing button, the operator selects means for activating a subcombination of any combination of LEDs 24, LED arrays 25, laser diodes 26, and laser diode arrays 27 disposed upon the headpiece 28 of the invention, which subcombination confines the emission of the LEDs 24, LED arrays 25, laser diodes 26, and laser diode arrays 27, to the pattern of hair loss represented by the icon on the selected button.

When combinations of LEDs 24, LED arrays 25, laser diodes 26, and laser diode arrays 27 are disposed upon an embodiment of the head covering for covering the human head 22, the resultant embodiment of the headpiece 28 of the invention may be directly applied to the scalp to administer LLL for the treatment of hair loss and the augmentation of hair retention. Alternatively, the headpiece 28 of the invention, may be disposed within a secondary head covering, such as a helmet, bonnet, cap, hood, or dome, to administer LLL to the scalp for the treatment of hair loss and the augmentation of hair retention.

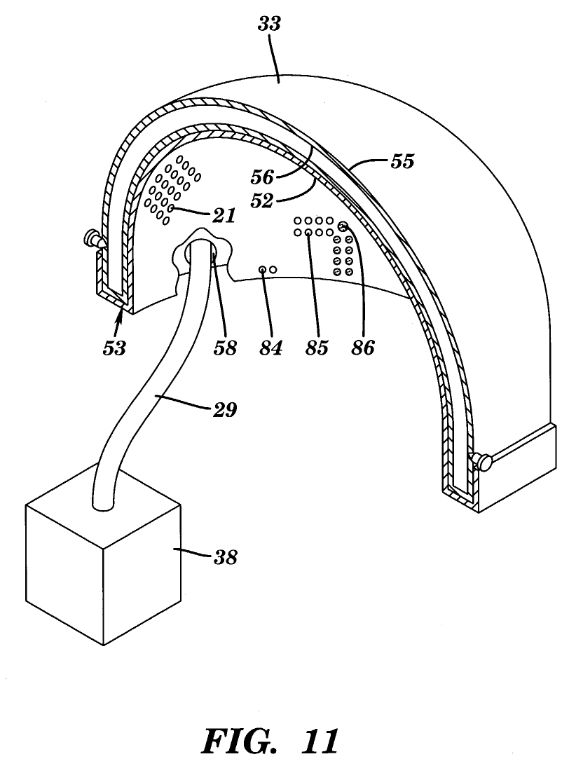

For example, FIG. 11, shows a perspective and sectional view of an embodiment of the head covering 22, shown in FIG. 1, in the form of a perforated inner hemispheric shell 52, disposed within an embodiment of a secondary head covering, in the form of a conforming outer imperforate shell 55, which is supported by a upturned rim 53 of the inner shell 52, thereby defining a space 56 between the inner 52 and outer 55 shells, and forming a hood 33.

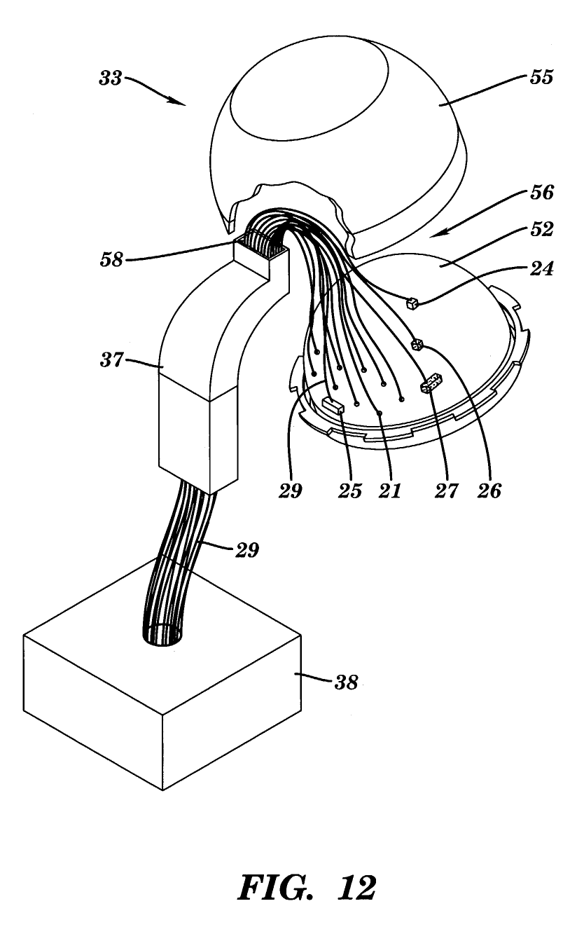

FIG. 11 further schematically illustrates an internal view of a disposition of a representative combination of light sources from an emitting face of a LED 84, an emitting face of a LED array 85, an emitting face of a laser diode 86, and an emitting face of a laser diode array 87. As shown in FIG. 12, the disposition of said representative combination of light sources corresponds to a disposition of a representative LED, 24, LED array 25, laser diode 26, and laser diode array 27, within the space 56 and upon the external surface of the perforated inner hemispheric shell 52 and within the space. In FIG. 11, the outer shell 55 is provided with an opening 58 to admit conductors 29, shown in FIG. 12, carried by a hollow, articulable and adjustable armature 37, from an embodiment of the control module 38, for distribution within the space 56, and thence for operable connection with the representative combination of a LED 24, LED array 25, laser diode 26, and laser diode array 27, disposed upon the external surface of the inner perforated inner hemispheric shell 52, coincident with the perforations 21 fashioned thereon.

The invention is also adaptable to variety of structural configurations.

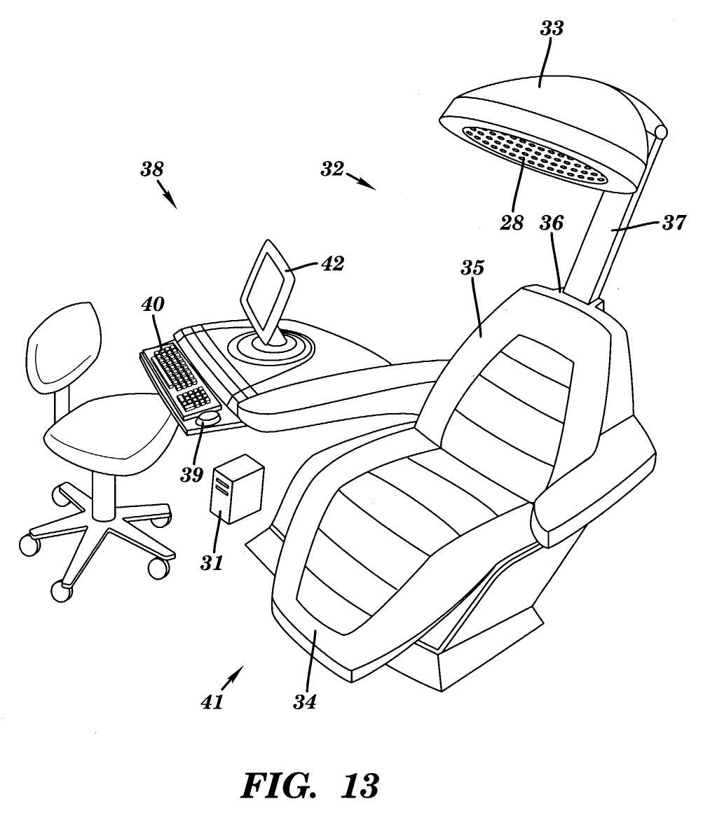

For purposes of illustration, FIG. 13 shows an embodiment of the invention in the form of a stationary LLLT station 41, comprising an embodiment of the control module 38, described supra and shown in FIG. 7 and FIG. 8, and a treatment module 32.

The treatment module 32 of the LLLT station 41 comprises a chair 34 whose back 35 bears mounting means 36 for mounting a hollow, articulable and adjustable armature 37, whose superior end pivotably connects to and supports an embodiment of a secondary head covering in the form of a hood 33, described supra., and shown in FIG. 11 and in FIG. 12.

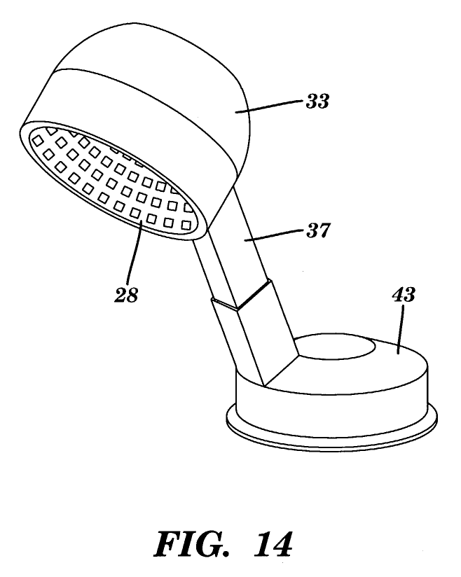

FIG. 14 shows a perspective view of a portable embodiment of the invention, wherein the superior aspect of the armature 37 is pivotably connected to a hood 33, and the inferior aspect of the armature 37 is pivotably connected to a portable base 43 adapted to contain, or operably connect to, an embodiment of the control module 38.

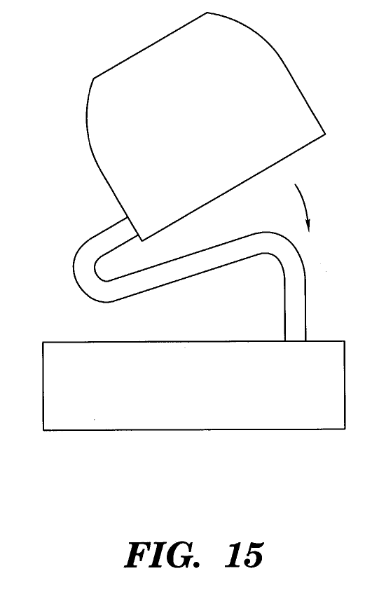

As shown in FIG. 15, in a portable embodiment, the invention may be folded from its operable position into its storage or carrying position in a manner well known in parallel mechanical arts.

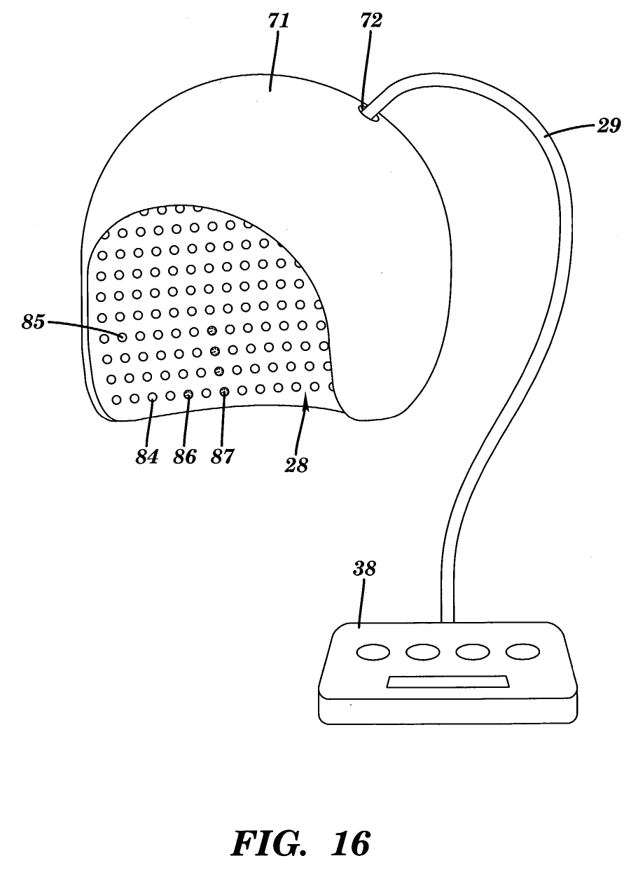

In another embodiment of the invention for the self-administration of LLLT, shown in FIG. 16, the headpiece 28 of invention is disposed within an embodiment of the secondary head covering in the form of a helmet 71, having an opening 72 to admit conductors 29 emanating from a hand-held embodiment of a control module 38, for operable distribution to and connection with a representative combination of a LED 84, a LED array 85, a laser diode 86, and a laser diode array 87, said representative combination being disposed upon the headpiece 28, within the helmet 71.

Accordingly, different embodiments of the invention may be used in different treatment settings and in different manners determined by different treatment protocols.

In one example of a use of the invention, a subject with regions of his or her scalp in need of hair retention or regeneration may be seated in the chair 34 of the LLLT station 41, shown in FIG. 13, with his or her head covered by the hood 33. An operator may then use an embodiment of the control module 38 to select LLL parameters within the module's control. For example, an operator may select the continuous mode of LLL emission, at a wavelength of about 670 nm, delivering an irradiance of about 500 mJ/cm², to be applied for about 15 minutes to one or more selected regions of the subject's scalp. The resulting application of LLL may comprise a treatment dose.

The treatment dose may be repeated at least once weekly, and the aggregate of said treatment doses given in the course of a week may comprise a weekly dose.

The weekly dose may be repeated over the course of about 1 to 52 consecutive weeks, and the aggregate of weekly doses may comprise a total dose distributed over a course of LLLT.

During a course of LLLT, the treatment parameters, treatment dose, weekly dose, total dose or duration of a course of LLLT may be altered depending upon the subject's response to the LLL produced by the LLL parameters previously selected.

A course of LLLT may be repeated periodically or applied to other regions of a subject's scalp in need of hair retention or regeneration.

Other courses of LLLT, governed by other protocols, may use other treatment doses and may use other weekly doses that are administered over other time periods. For example, a course of maintenance LLLT may be administered pursuant to a protocol designed to maintain the benefit of an earlier administered course of LLLT.

In another example of a use of the invention, a subject with regions of his or her scalp in need of hair retention or regeneration may use a portable embodiment of the invention, as shown in FIG. 14 or in FIG. 16, for the self-administration of LLL, selecting LLL parameters from a portable embodiment of the control module 38 pursuant to a protocol provided by a hair retention and regeneration practitioner.

The foregoing description of embodiments and uses of this invention has been presented for purposes of illustration and description. It is not intended to be exhaustive or to limit the invention to the precise form or use disclosed, and obviously, many modifications and variations are possible. Such modifications and variations that may be apparent to a person skilled in the art are intended to be included within the scope of this invention as defined by the accompanying claims.

I claim:

1. An apparatus, comprising:
a head covering for covering a human head; and
a light source operatively connected to said head covering, wherein said light source is directed toward the scalp of said human head when said head covering is worn upon said head.

2. The apparatus of claim 1, wherein said light source comprises a light emitting diode.

3. The apparatus of claim 1, wherein said light source comprises a laser diode.

4. The apparatus of claim 1, wherein said light source comprises a light emitting diode array.

5. The apparatus of claim 1, wherein said light source comprises a laser diode array.

6. The apparatus of claim 1 wherein said light source is chosen from the group consisting of light emitting diodes, light emitting diode arrays, laser diodes, laser diode arrays, and any combination thereof.

7. The apparatus of claim 1, wherein said light source emits light having a wavelength between about 400 nm and about 1000 nm.

8. The apparatus of claim 1, wherein said light source emits light having a wavelength of about 670 nm.

9. The apparatus of claim 1, wherein said light source delivers an irradiance lying between about 100 mJ/cm² and about 900 mJ/cm².

10. The apparatus of claim 1, wherein said light source is operationally connected to means for selectively exposing selected locations, areas, and patterns of said scalp of said human head to light emitted from said light source.

11. The apparatus of claim 10, wherein said locations, areas, and patterns of said scalp of said human head selected for exposure to light emitted from said light source, correspond to icons of a set of icons, each depicting a pattern of hair loss replicating a state of hair loss of the human head.

12. The apparatus of claim 11, wherein said icons of said set of icons, each depict a pattern of hair loss replicating a state of hair loss of the Hamilton scale.

13. The apparatus of claim 11, wherein said icons of said set of icons, each depict a pattern of hair loss replicating a state of hair loss of the Ludwig scale.

14. The apparatus of claim 11, wherein said icons of said set of icons, each depicting a pattern of hair loss replicating a state of hair loss of said human head, appear as tiles on the screen of a computer viewing device, the touching of which by a computer pointing device executes means for activating an emission from said light source corresponding to the state of hair loss depicted by said icon on said tile.

15. The apparatus of claim 11, wherein said icons of said set of icons, each depicting a pattern of hair loss replicating a state of hair loss of said human head, appear on keys of a computer keyboard, the striking of which executes means for activating an emission from said light source corresponding to the state of hair loss depicted by said icon on said key.

16. The apparatus of claim 11, wherein said icons of said set of icons, each depicting a pattern of hair loss replicating a state of hair loss of said human head, appear on buttons disposed upon a surface, the pressing of which executes means for activating an emission from said light source corresponding to the state of hair loss depicted by said icon on said button.

17. The apparatus of claim 1, wherein said light source is operationally connected to means for variably exposing said selected locations, areas, and patterns of the scalp of said human head to light emitted from said light source over time.

18. The apparatus of claim 1, wherein said light source is operationally connected to means for emitting light from said light source in a continuous mode.

19. The apparatus of claim 1, wherein said light source is operationally connected to means for emitting light from said light source in a pulsed mode.

20. The apparatus of claim 19, wherein said light source is operationally connected to means for varying the pulse frequency of said pulsed mode.

21. The apparatus of claim 1, wherein said light source is operationally connected to means for selecting the emission of light in either a continuous emission or a pulsed emission.

22. The apparatus of claim 1, further comprising a photoelectric cell operationally connected to means for deactivating said light source whenever said head human head is withdrawn from said head covering.

23. The apparatus of claim 1, wherein said head covering has a shape conforming to a human head.

24. The apparatus of claim 1, wherein said head covering is a wireframe mesh.

25. The apparatus of claim 1, wherein said head covering is formed from plastic.

26. The apparatus of claim 1, wherein said head covering is a helmet.

27. The apparatus of claim 1, wherein said head covering is a hat.

28. The apparatus of claim 1, wherein said head covering is a cap.

29. The apparatus of claim 1, wherein said head covering is a hood.

30. The apparatus of claim 1, wherein said head covering is a dome.

31. The apparatus of claim 1, wherein said head covering is a cowl.

32. The apparatus of claim 1, wherein said head covering is a handkerchief.

33. The apparatus of claim 1, wherein said head covering is disposed within means for covering said human head.

34. The apparatus of claim 33, wherein said means for covering said human head is a helmet.

35. The apparatus of claim 33, wherein said means for covering said human head is a hat.

36. The apparatus of claim 33, wherein said means for covering said human head is a cap.

37. The apparatus of claim 33, wherein said means for covering said human head is a hood.

38. The apparatus of claim 33, wherein said means for covering said human head is a dome.

39. The apparatus of claim 33, wherein said means for covering said human head is a cowl.

40. The apparatus of claim 33, wherein said means for covering said human head is a handkerchief.

41. A method of regenerating and retaining hair comprising a single application of LLL to at least one region of the scalp of a human head, said application of LLL being emitted from a light source is chosen from the group consisting of light-emitting diodes, light-emitting diode arrays, laser diodes, laser diode arrays and any combination thereof, said light source being disposed upon a head covering for covering a human head, and said light source being operationally connected to means for selecting regions of the scalp in need of hair retention or regeneration to be exposed to LLL, means for selecting LLL wavelengths, means for selecting LLL exposure durations, means for selecting LLL irradiances, and means for selecting the emission of either continuous or pulsed LLL.

42. The method of claim 41, wherein said single application of said LLL uses LLL that is continously emitted from said light source for a continuous temporal duration ranging from about 1 minutes to about 30 minutes to produce a single dose of continuous LLL.

43. The method of claim 41, wherein said single application of said LLL uses LLL that is emitted from said light source in pulses of said LLL, said pulses having a temporal duration ranging from about 100 milliseconds to about 1 milliseconds and having a corresponding frequency ranging from about 10 Hertz to about 1000 Hertz.

44. The method of claim 43, wherein said pulses of LLL emitted from said light source are emitted over a continuous temporal duration in the range between about 1 minutes to about 30 minutes to produce a single dose of pulsed LLL.

45. The method of claim 41, wherein said single application of LLL is repeated.

46. The method of claim 41, wherein said single application LLL is confinable to said region of the scalp in need of hair retention or regeneration.

47. The method of claim 41, wherein said LLL has a wavelength between about 400 nm and about 1000 nm.

48. The method of claim 41, wherein said LLL delivers an irradiance between about 100 mJ/cm² and about 900 mJ/cm².

49. The method as in claim 41, wherein said region of the scalp in need of hair retention or regeneration is selectable.

50. The method of claim 41, wherein said region of the scalp in need of hair regeneration or retention is selected to correspond to stages of hair loss comprising the Hamilton scale.

51. The method of claim 41, wherein said region of the scalp in need of hair regeneration or retention is selected to correspond to stages of hair loss comprising the Ludwig scale.
